SEQUENCE LISTING (9 sequences)

SEQ ID NO: 1 - DNA, 19 bp, synthetic construct
  source 1..19: mol_type = other DNA
ctctgccacccaacacgag

SEQ ID NO: 2 - protein, 111 aa, synthetic construct
DIVLTQSPASLAVSLGQRATISCRASESIEYYGTSLMQWYQQKPGQPPKLLIYAASNVESGVPARFSGSGSGTDFSLNIHPVEEDDIAMYFCQQSRKVPTFGGGTKLEIKR

SEQ ID NO: 3 - protein, 123 aa, synthetic construct
EVQRVESGPGLVKPSQSLSLTCTVTGYSITNDYAWNWIRQFPGNKLEWMGYISYSGSTSYNPYLKSRISITRDTSKNQFFLQLNSVPTEDTATYYCARSTYGNYGYYYAMDCWGQGTSVTVSS

SEQ ID NO: 4 - protein, 108 aa, synthetic construct
DIVMTQSPSSLSASLGERVSLTCRASQEISGYLSWLQQKPDGTIKRLIYAASTLDSGVPKRFSGSRSGSDYSLTISSLESEDFADYYCLQYASYPWTFGGGTKLEIKR

SEQ ID NO: 5 - protein, 113 aa, synthetic construct
QVQLQQPGSELVRPGASVKLSCKASGYTFTSYWMHWVKQRHGQGLEWIGNIYPGSGSTNYDEKFKSKGTLTVDTSSSTAYMHLSSLTSEDSAVYYCTTGTAYWGQGTLVTVSA

SEQ ID NO: 6 - protein, 20 aa, synthetic construct
RGPSLPTPPGREPKKVAVVR

SEQ ID NO: 7 - DNA, 26 bp, synthetic construct
  source 1..26: mol_type = other DNA
acgctattaactattaacgtacctgt

SEQ ID NO: 8 - DNA, 26 bp, synthetic construct
  source 1..26: mol_type = other DNA
cgaagcgcagtaaggatggctagtgt

SEQ ID NO: 9 - DNA, 22 bp, synthetic construct
  source 1..22: mol_type = other DNA
atattgcagcagtacgcacaca

(19) United States
(12) Patent Application Publication
(10) Pub. No.: US 2023/0296555 A1
(43) Pub. Date: Sep. 21, 2023

(54) METHOD FOR PREPARING BIOCHIP AND BIOCHIP PREPARED THEREFROM

(71) Applicant: NOVASCOPE BIOCHIPS INC., Dover, DE (US)

(72) Inventors: CHANG-FU KUO, Taipei City (TW); LIAN-CHIN WANG, Taipei City (TW); AO-HO HSIEH, Taipei City (TW); MING-TANG CHIOU, Taipei City (TW); WEI-HAO LIN, Taipei City (TW)

(21) Appl. No.: 18/147,716

(22) Filed: Dec. 29, 2022

Related U.S. Application Data
(60) Provisional application No. 63/321,209, filed on Mar. 18, 2022.
(60) Provisional application No. 63/321,211, filed on Mar. 18, 2022.
(60) Provisional application No. 63/336,546, filed on Apr. 29, 2022.
(60) Provisional application No. 63/347,570, filed on Jun. 1, 2022.

(51) Int. Cl. G01N 27/414; C12Q 1/6825; G01N 33/543

(52) U.S. Cl. CPC G01N 27/4145; C12Q 1/6825; G01N 33/5438; C12Q 1/701

(57) ABSTRACT

The present disclosure provides a method for preparing a biochip. The method includes steps of coating a chip with a first solution of biotin to form a biotin-coated chip, wherein the biotin in the first solution is in a first concentration ranging from 0.1 to 1 μg/ml; providing the biotin-coated chip with a second solution of avidin to form an avidin/biotin-coated chip, wherein the avidin in the second solution is in a second concentration ranging from 0.1 to 100 μg/ml; and providing the avidin/biotin-coated chip with a third solution of a biotinylated probe to form the biochip, wherein the biotinylated probed in the third solution is in a third concentration ranging from 1 to 3 μg/ml.

### CROSS-REFERENCE TO RELATED APPLICATION

This non-provisional application claims priority under 35 U.S.C. § 119(e) on U.S. Provisional Patent Application No. 63/321,211 filed on Mar. 18, 2022, U.S. Provisional Patent Application No. 63/321,209 filed on Mar. 18, 2022, U.S. Provisional Patent Application No. 63/336,546 filed on Apr. 29, 2022, and U.S. Provisional Patent Application No. 63/347,570 filed on Jun. 1, 2022, and the entire contents of these applications are hereby incorporated by reference.

### REFERENCE TO AN ELECTRONIC SEQUENCE LISTING

The content of the electronic sequence listing (sequencelisting.xml; size: 9,138 bytes; and date of creation: Dec. 7, 2022) is herein incorporated by reference in its entirety.

### BACKGROUND OF THE INVENTION

### 1. Field of the Invention

The present disclosure relates to a method for preparing a biochip, and in particular to a method for preparing a biochip with improved sensitivity and a biochip prepared therefrom.

### 2. Description of the Related Art

A chip is formed of an integrated circuit (IC) consisting of transistors, capacitors, and other electronic components on a small flat piece of semiconducting material, and used to process electronic signals for various measurements.

One of the common transistors used in the chip is the metal oxide semiconductor field-effect transistor (MOSFET) that controls the current flow through an electric field. The MOSFET has the advantages such as high sensitivity, high specificity, and real-time detection.

A biochip includes a biological element and the chip element. The surface of the biochip needs to be modified to accommodate a specific probe for various detection targets.

The (3-aminopropyl) triethoxy silane (APTES) and glutaraldehyde (GA) are the most common modifier and activating agents for surface functionalization of the chip. APTES is an amine-functionalized organosilane. GA is a dialdehyde that contains two aldehyde functional groups. The amine group from APTES can easily react with the aldehyde group from GA. One of the aldehyde groups of GA can interact with APTES, and the other aldehyde group interacts with the probe's antibodies (Udomsom et al., Coatings (2021), 11, 595).

Other modifiers such as CPTES, VTES, and activating agents such as gelatin, EDC, and NHS can also be applied to the grafting process (Kujawa et al., Science of the Total Environment 801 (2021) 149647). Triethoxysilylundecanal (TESUD) is an alternative grafting agent which can replace the two-step of APTES-GA (Lee et al., Sensors and Actuators B 175 (2012) 201-207).

Due to functionalization of the surface of the bio-FET chip, functional groups are attached to the surface for antibody probe binding, and then the analytes are added for detection (Sadighbayan et al., Trends in Analytical Chemistry (2020)).

However, the number of functional groups carried by each antibody probe is various, which may lead to inconsistent binding efficiency. Furthermore, the orientation of antibody probes binding to the APTES or GA is random, which may reduce the number of Fab (fragment, antigen-binding) regions interactive with antigens and decrease the antibody probe density (Trilling et al., Analyst (2013)). Biotin/avidin affinity system has been commonly used to improve this issue. Although this method can increase the binding efficiency of the probes, the sensitivity is still low.

Thus, it is desirable to prepare a biochip having biotin/avidin/biotinylated probe with improved sensitivity.

### BRIEF SUMMARY OF THE INVENTION

It is an object of the present disclosure to provide a biochip with significantly improved sensitivity.

To achieve at least the above object, the present disclosure provides a method for preparing a biochip, including the steps of coating a chip with a first solution of biotin to form a biotin-coated chip, wherein the biotin in the first solution is in a first concentration ranging from 0.1 to 1 μg/ml; providing the biotin-coated chip with a second solution of avidin to form an avidin/biotin-coated chip, wherein the avidin in the second solution is in a second concentration ranging from 0.1 to 100 g/ml; and providing the avidin/biotin-coated chip with a third solution of a biotinylated probe to form the biochip, wherein the biotinylated probed in the third solution is in a third concentration ranging from 1 to 3 μg/ml.

In an embodiment, the chip is an extended gate field-effect transistor (EGFET).

In an embodiment, the biotin in the first solution is in the first concentration of 0.1 μg/ml.

In an embodiment, the avidin in the second solution is in the second concentration of 30 μg/ml.

In an embodiment, the biotinylated probed in the third solution is in the third concentration of 1 μg/ml.

In an embodiment, the biotin in the first solution is in the first concentration of 1 μg/ml.

In an embodiment, the avidin in the second solution is in the second concentration of 100 μg/ml.

In an embodiment, a ratio of the first concentration, the second concentration and the third concentration is 1:300:10.

In an embodiment, the biotinylated probe is selected from the group consisting of a biotinylated PRRSV antibody, a biotinylated anti-Tau antibody and a biotinylated SARS-CoV-2 probe.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A are images taken via the confocal microscope showing the fluorescence intensity of biotin-FITC coated on the chip.

FIG. 1B are images taken via the confocal microscope showing the fluorescence intensity of biotin-FITC coated on the chip.

FIG. 2 are images taken via the confocal microscope showing the fluorescence intensity of Avidin Alexa Fluor 488 coated on the chip.

FIG. 3A are images taken via the confocal microscope showing the fluorescence intensity of Avidin Alexa Fluor 488 on the chip which has been treated with 100 ng/ml of biotin-NHS.

FIG. 3B are images taken via the confocal microscope showing the fluorescence intensity of Avidin Alexa Fluor 488 on the chip which has been treated with 1 μg/ml of biotin-NHS.

FIG. 4A are images taken via the confocal microscope showing the fluorescence intensity of PRRSV probe-FAM on the chip which has been treated with 100 ng/ml of biotin and then 30 μg/ml of avidin.

FIG. 4B are images taken via the confocal microscope showing the fluorescence intensity of PRRSV probe-FAM on the chip which has been treated with 1 μg/ml of biotin and then 100 μg/ml of avidin.

FIG. 5A is a diagram showing the electrical signals of 1 ng/ml, 1 μg/ml, and g/ml avidin, respectively, on the chip which has been treated with 0.1 μg/ml biotin.

FIG. 5B is a diagram showing the electrical signals of 0.1 μg/ml, 1 μg/ml, and 3 μg/ml of biotinylated antibody, respectively, on the chip which has been treated with biotin and avidin.

FIG. 6 is a diagram showing validation of concentration range of Tau peptide on the chip.

FIG. 7 is a diagram showing validation of concentration range of SARS-CoV-2 RNA on the chip.

### DETAILED DESCRIPTION OF THE INVENTION

To facilitate understanding of the object, characteristics and effects of this present disclosure, embodiments together with the attached drawings for the detailed description of the present disclosure are provided.

The chip in this disclosure is an extended gate field-effect transistor (EGFET) for sensing analytes (e.g., biological analytes). A biosensing system can utilize a bio-detection layer on a substrate (e.g., a conductive substrate or sensing surface area), which can be coupled to a field effect transistor (FET). The gate of the field effect transistor is connected to the substrate having the bio-detection layer thereon. The functionalized substrate can include a well-defined area (e.g., a well, matrix or a flat area) that can hold a specific, pre-determined volume of fluid (e.g., liquid, colloid) on it. An external electrode can be dipped in the fluid and can then be connected to a power source supplying a voltage, which can be the gate voltage or power source. The variations in the charge transfer from the external electrode to the gate electrode of the FET due to the presence of target molecules on the bio-detection layer modulates the source to drain characteristics of the FET. These modulations can be then correlated to the concentration of the target analyte (e.g., based on an already known correlation due to a prior calibration or the like) that is present on the detection layer. Such a biosensing system allows for the isolation of the sensitive electronic components from the potentially detrimental environment of the fluids used for sensing (i.e., the FET components are isolated from the fluid containing the analyte). This system can include an external functionalized substrate that is connected to the gate of the FET. The FET also includes a source and a drain, along with the insulator and gate electrode of the gate. The external electrode can touch the electrolyte, and a gate voltage is applied across the external electrode. The interaction between the ions present in the electrolyte and the functional group present on the extended gate substrate causes a surface potential change. This change of potential causes a change in the flow of current to the gate of the FET. In turn, the gate current modulates the drain-source current in the FET. The modulation of drain current due to the presence of biomolecules on the surface of the extended gate makes the FET as a biosensor.

Determination of Coating Concentration of Biotin Via Confocal Microscopy

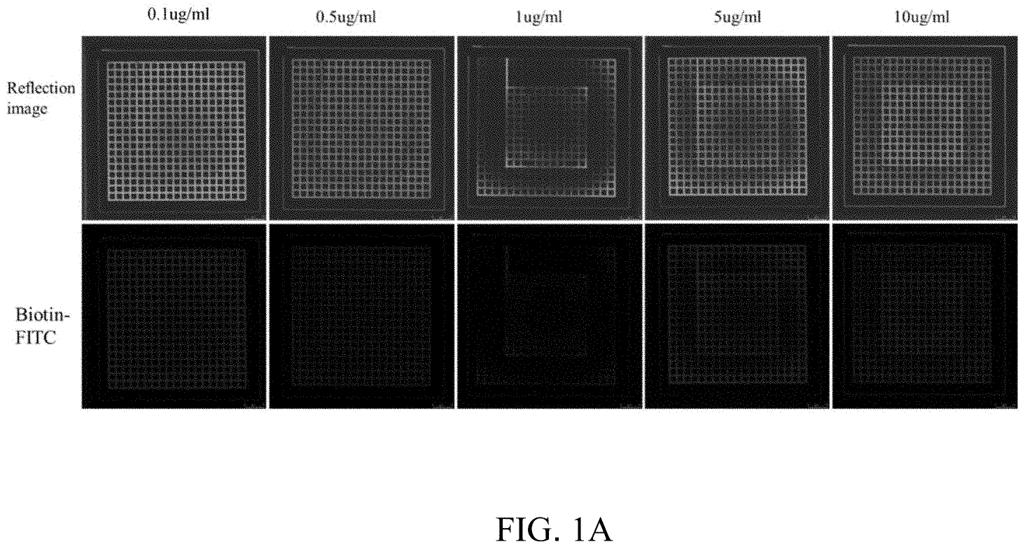

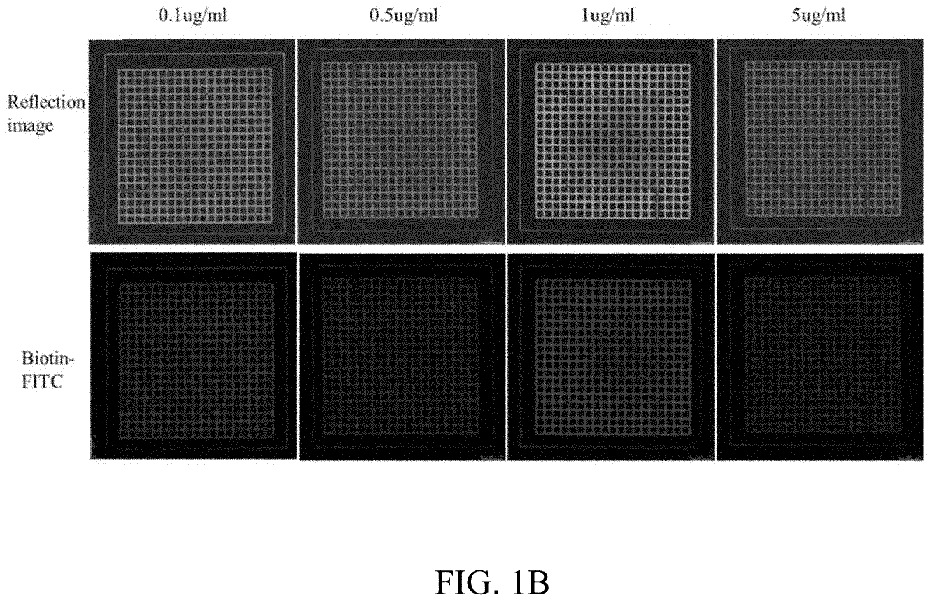

An EGFET was treated with the biotin coupled to fluorescein isothiocyanate (FITC) (Thermo Scientific, #22030) at concentrations of 0.1 μg/ml, 0.5 μg/ml, 1 g/ml, and 10 μg/ml, respectively, in phosphate-buffered saline (PBS) solution for 16 hours to form a biotin-coated chip. Then, the fluorescence intensity was observed using a confocal microscope (TCS SP8 STED, Leica), and the images were shown in FIG. 1A. However, the surface of the biotin-coated chip was not flat, leading to difficulty in focusing. Therefore, the confocal image of the biotin-coated chip treated with 1 μg/ml of biotin-FITC was taken again. The results shown in FIG. 1B indicate that the fluorescence intensity was strong at concentrations of 0.1 μg/ml and 1 μg/ml. In consideration of cost of material, the optimal concentration of biotin to be coated onto the chip is 0.1 μg/ml.

Determination of Appropriate Concentration Range of Avidin

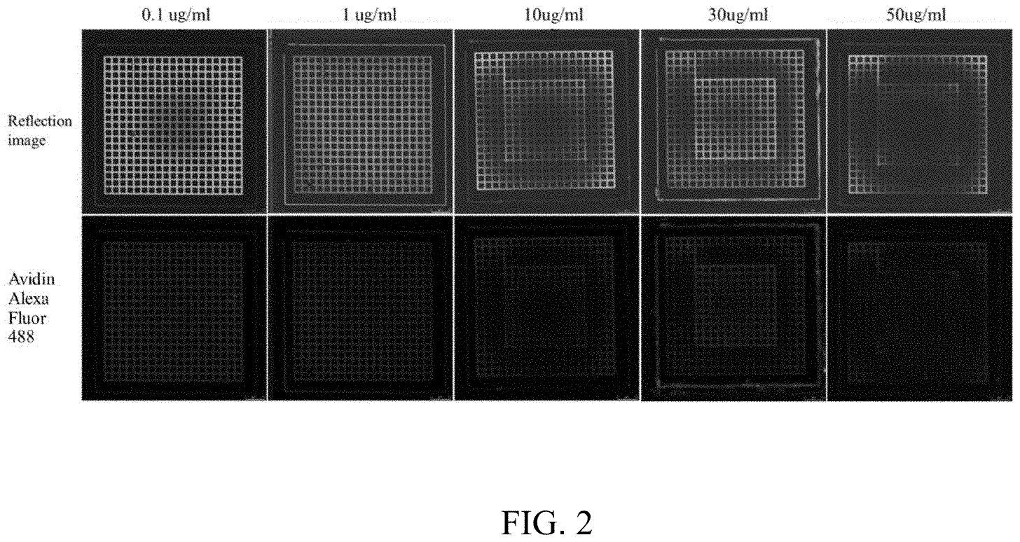

An EGFET was treated with the Avidin Alexa Fluor 488 (Thermo Scientific, #A21370) at concentrations of 0.1, 1, 10, 30, and 50 μg/ml, respectively, in phosphate-buffered saline (PBS) solution for 10 minutes to form an avidin-coated chip. Then, a confocal microscope was used to observe the fluorescence intensity of the avidin-coated chips, and the images were shown in FIG. 2. The results shown in FIG. 2 indicate that the fluorescence intensity was strong at concentrations of 0.1 g/ml and 30 μg/ml.

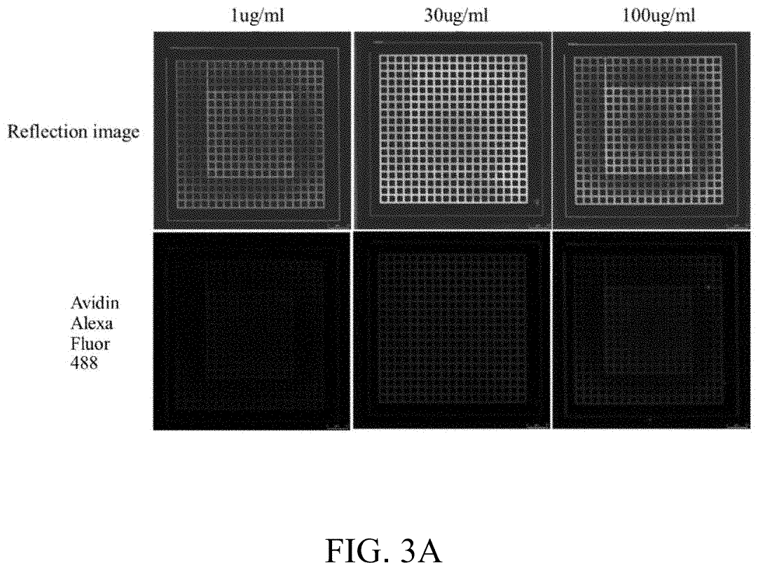

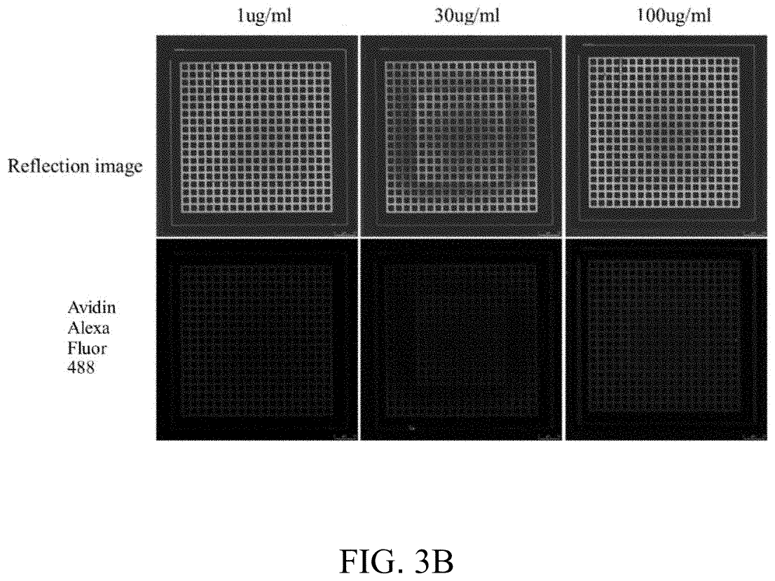

Subsequently, an EGFET was treated with biotin-NHS (Thermo Scientific, #20217) at the concentration of 0.1 μg/ml to form a biotin-coated chip, and then the biotin-coated chip was treated with Avidin Alexa Fluor 488 at the concentrations of 1, 30 and 100 μg/ml, respectively, for 10 minutes to form an avidin/biotin-coated chip. Then, a confocal microscope was used to observe the fluorescence intensity of the avidin/biotin-coated chips, and the images were shown in FIG. 3A. 30 μg/ml of avidin is equivalent to 2.7×10\hat{ }14 molecules of avidin, and 0.1 μg/ml of biotin is equivalent to 2.4×10\hat{ }14 molecules of biotin, that is, the ratio of avidin molecules to biotin molecules is 1:1. The results shown in FIG. 3A indicate that after the biotin-coated chips (treated with 0.1 μg/ml of biotin) were treated with Avidin Alexa Fluor 488 at the concentrations of 1, 30 and 100 μg/ml, respectively, to form the avidin/biotin-coated chips, the fluorescence intensity of avidin/biotin-coated chip was optimal at the avidin concentration of 30 μg/ml. In contrast, after the biotin-coated chips (treated with 1 μg/ml of biotin) were treated with Avidin Alexa Fluor 488 at the concentrations of 1, 30 and 100 μg/ml, respectively, to form the avidin/biotin-coated chips, the fluorescence intensity of avidin/biotin-coated chip was optimal at the avidin concentration of 100 μg/ml, as shown in FIG. 3B. 100 μg/ml of avidin is equivalent to 9×10\hat{ }14 molecules of avidin, and 1 μg/ml of biotin is equivalent to 2.4×10‥molecules of biotin, that is, the ratio of avidin molecules to biotin molecules is 1:2.66. 30 μg/ml of avidin is equivalent to 2.7×10\hat{ }14 molecules of avidin, and 1 μg/ml of biotin is equivalent to 2.4×10‥molecules of biotin, that is, the ratio of avidin molecules to the biotin molecules is 1:8.88. In FIG. 3B, the fluorescence intensity resulting from 1 μg/ml of biotin and 100 μg/ml of avidin is better than that resulting from 1 μg/ml of biotin and 30 μg/ml of avidin.

Determination of Appropriate Concentration Range of Biotinylated Probe

In light of the above experiments, the combination of 0.1 μg/ml of biotin and 30 μg/ml of avidin and the combination of 1 μg/ml of biotin and 100 μg/ml of avidin were used respectively for the subsequent experiment to determine the optimal concentration of the biotinylated probe.

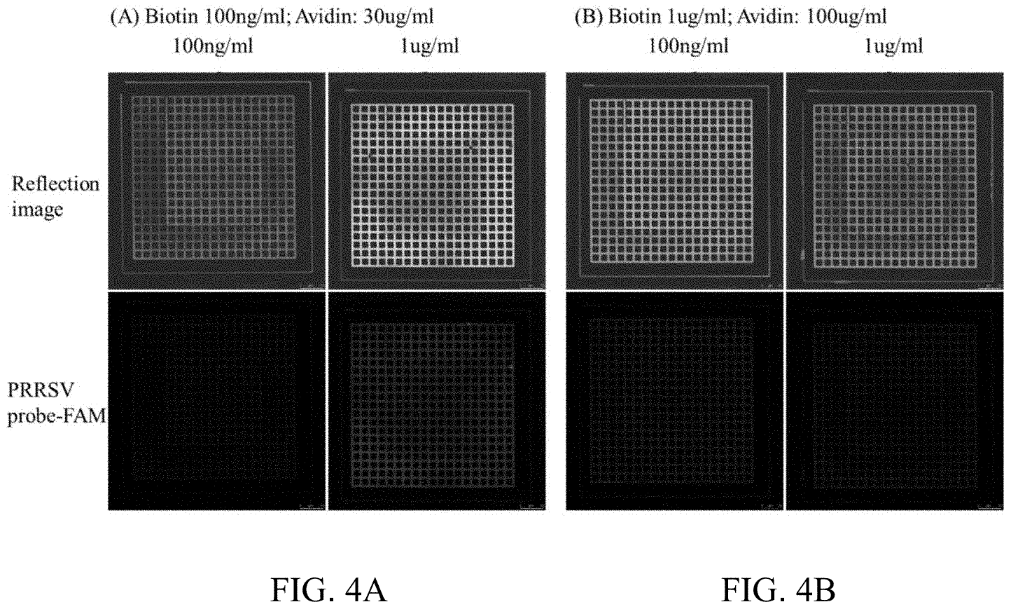

The nucleic acid probe was modified to have biotin at the 5′end and to have fluorescein amidites (FAM) at the 3′ end. It was observed that the chip coated with 0.1 μg/ml of biotin, 30 μg/ml of avidin, and then 1 μg/ml of PRRSV (Porcine Reproductive and Respiratory Syndrome Virus) probe (SEQ ID NO: 1) had the optimal fluorescence intensity, as shown in FIG. 4.

Validation of Concentration Range of Biotinylated Antibody on Chip

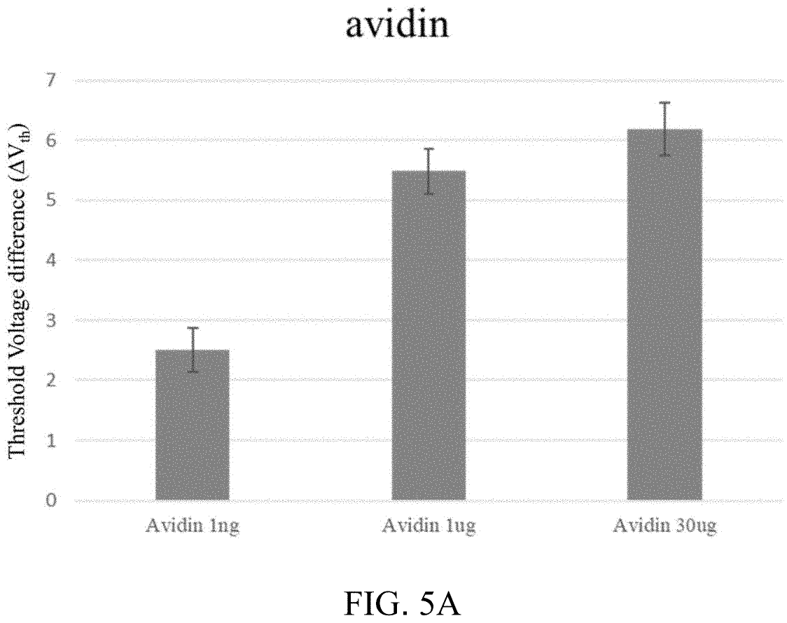

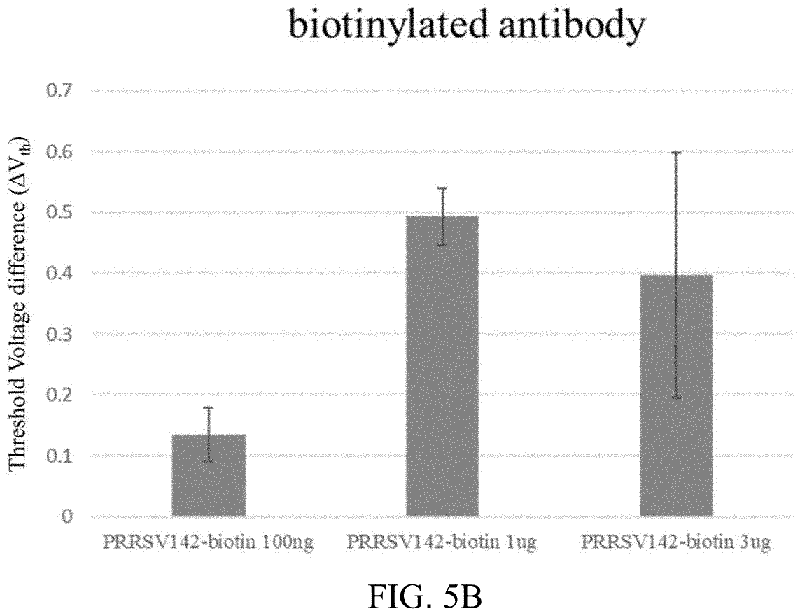

The concentration conditions obtained above were verified based on the changes in electrical signals. The variations in the electrical signals of the chip coated with 0.1 μg/ml of biotin and 30 μg/ml of avidin were measured at various concentrations, 0.1 μg/ml, 1 μg/ml and 3 μg/ml, of biotinylated PRRSV antibody including PRRSV-142 VL (SEQ ID NO: 2) and PRRSV-142 HL (SEQ ID NO: 3). The threshold voltages (Vₜₕ) of the ID-VG curves of each concentration were obtained at a constant drain current. Threshold voltage changes (ΔVₜₕ) were obtained by calculating the difference between the threshold voltages of the baseline and each concentration. As the avidin concentration increased to 30 μg/ml, ΔVₜₕ increased, as shown in FIG. 5. As the avidin concentration increased to 30 μg/ml, ΔVₜₕ; increased to 6.19 1 μg/ml of biotinylated PRRSV probe yields the maximum ΔVₜₕ value at 0.49. However, when the concentration of biotinylated PRRSV probe increased to 3 μg/ml, ΔVₜₕ not only reduced but also became unstable.

Validation of Concentration Range of Target Peptide on Chip

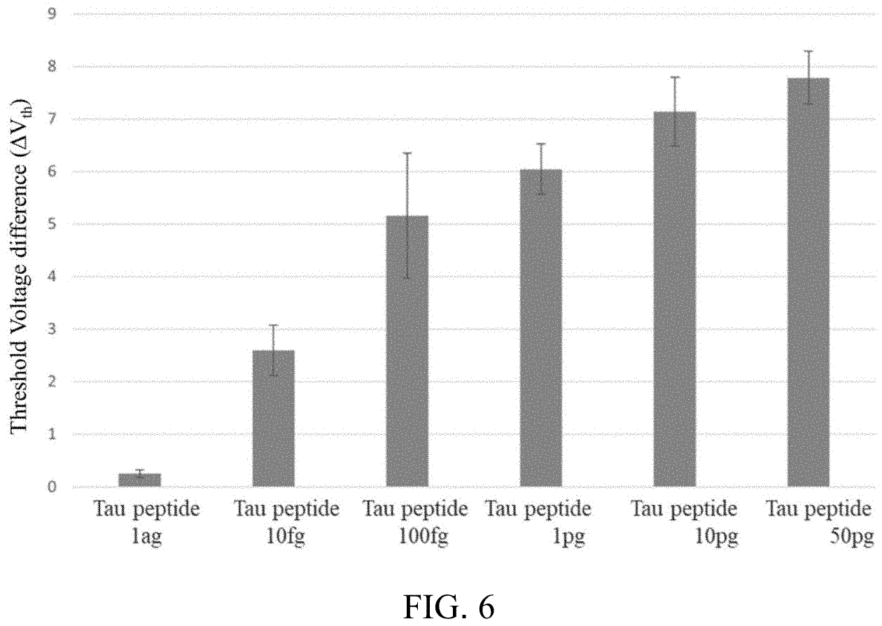

Biotinylated anti-Tau antibody was initially used to measure the detectable range of the target peptide to determine the sensitivity and detectable range of pathogens in this biological system. The chip was coated with 0.1 μg/ml of biotin, g/ml of avidin, and with 1 μg/ml of the biotinylated Tau antibody including Tau-217-123 VL (SEQ ID NO: 4) and Tau-217-123 VH (SEQ ID NO: 5), and subsequently, the various concentrations of the target peptide (i.e., Tau peptide (SEQ ID NO:6)) were added, respectively. The results indicated that the lowest detectable concentration of Tau peptide was 1 ag/ml, and the highest detectable concentration of Tau peptide was 50 pg/ml on the chip, as shown in FIG. 6.

Validation of Concentration Range of Target Nucleic Acid on Chip

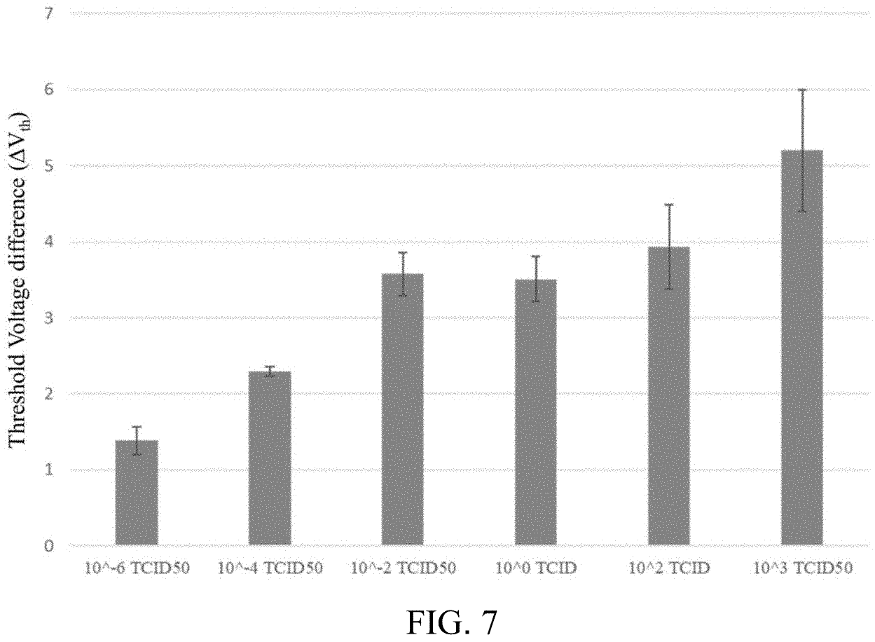

A biotinylated SARS-CoV-2 probe including probe-F (SEQ ID NO:7), probe-P1 (SEQ ID NO:8) and probe-R (SEQ ID NO:9) in a ratio of 1:1:1 was used to test the sensitivity of the detected pathogens on this biological system. The chip was coated with 0.1 μg/ml of biotin, 30 μg/ml of avidin, and then 1 μg/ml of the biotinylated SARS-CoV-2 probe. The original concentration of the SARS-CoV-2 total RNA is 2.68×10⁶ TCID50. The different concentrations, 2.68×10⁻⁶, 2.68×10⁻⁴, 2.68×10⁻², 2.68, 2.68×10² and 2.68×10³ TCID50, of the SARS-CoV-2 total RNA were then added. The results showed that the sensitivity of the chip for detecting SARS-CoV-2 total RNA reached 10⁻⁶ TCID50 (Ct:48) and exhibited a linear range from 10⁻⁶ TCID50 to 10³ TCID50, as shown in FIG. 7.

In the present disclosure, the optimal concentration of biotin, avidin and biotinylated PRRSV antibody used for coating the chip was 0.1, 30, and 1 μg/ml, respectively. Also, the optimal concentration ratio of biotin, avidin and biotinylated PRRSV antibody is 1:300:10. In addition, this ratio is also validated on the chip and can produce the maximum voltage signal value. This ratio was applied to detect p-Tau 217 and SARS-CoV-2.

Phosphorylated Tau protein in cerebrospinal fluid (CSF) is a vital biomarker of Alzheimer's disease (AD) (Garcia-Chamd et al., Iiosensors and Bioelectronics 159 (2020)). P-Tau 181, p-Tau 217, and p-Tau 231 are the major biomarkers for AD pathology (Leuzy et al., EMBO Mol Med (2022) 14: e14408). P-Tau217 has shown better performance than P-Tau 181 for AD early detection in plasma (Janelidze et al., Nature Communications (2020) 11:1683). The enzyme-linked immunosorbent assay (ELISA) is the most widely used immunoassay in biomarker studies. However, the relatively low sensitivity causes missing detection. Ultrasensitive assays such as electrochemiluminescence (ECL) immunosensor, single-molecule array (SIMOA), immunomagnetic reduction assay (IMR), and mass spectrometry (MS)-based techniques are suitable for plasma Tau protein detection (Park et al., Clin Neurol (2022) 18(4):401-409). SIMOA is currently the most sensitive detection method. The lower limit of quantification (LLOQ) of p-Tau217 in plasma by SIMOA assay is 1.8 pg/ml. The LLOQ of p-Tau217 in plasma by ECL-based immunosensor is 34 pg/ml and a detection range from 0.1 to 100 ng/ml (Jalili et al., Molecules (2022) 27, 431). The sensitivity of the p-Tau217 ELISA kit is 1 pg/ml to 80 pg/ml (OmnimAbs. OM641673).

In comparison with all techniques mentioned in the previous paragraph, the sensitivity of the biotin/avidin chip system of the present disclosure is more than 180-fold higher than the SIMOA system, and more than 340-fold higher than the ECL-based system. The limit of detection (LOD) of the biotin/avidin chip system is 1 ag/ml (equal to 0.001 fg/ml) and exhibits a linear range from 10 fg/ml to 50000 fg/ml. The average concentration of the p-Tau217 in the normal control plasma is 0.13 pg/ml (equal to 130 fg/ml) (Barthelemy et al., J. Exp. Med. (2020) Vol. 217 No. 11) which is well within the linear range detections of the current biotin/avidin chip system. In addition, this system needs only a small amount of plasma for detection and the operation procedure is relatively easy. Thus, the present disclosure provides a useful method and tool for p-Tau217 detection during the early stages in plasma for AD diagnosis.

For SARS-CoV-2 detection, real-time PCR is a gold standard method for the qualitative and quantitative detection of viral nucleic acids. However, this method needs to operate in qualified laboratories, skilled technical experts, and an extended period before analyzing data. Due to the pandemic of COVID-19 outbreak, many new detection methods have been developed, such as graphene-FET biosensors and silicon nanowire-based viral spike protein detection. The graphene-FET biosensor device could detect 16 pfu/ml in a culture medium and 2.42×10² copies/ml in clinical samples (Sadighbayan et al., Trends in Analytical Chemistry (2020)). The titer of 0.7 pfu can be estimated as theoretically equivalent to 1 TCID50 (Pourianfar et al., Indian J Virol. (2012)). Therefore, the 16 pfu/ml was equivalent to 22.8 TCID50. The low limit of detection of the current biotin/avidin chip system is 10⁻⁶ TCID50 (Ct:48). The sensitivity of the biotin/avidin chip system of the present disclosure is better than the graphene-FET biosensor. The biotin/avidin chip system of the present disclosure used nucleic acid as a probe for nucleic acid detection without amplification, which can shorten the detection time and does not require skilled technical experts. The biotin/avidin chip system of the present disclosure can quickly and precisely detect SARS-CoV-2 when the amount of virus is very low and still in the incubation period, which can be monitored early and immediately.

While the present disclosure has been described by means of specific embodiments, numerous modifications and variations could be made thereto by those skilled in the art without departing from the scope and spirit of the present disclosure set forth in the claims.

What is claimed is:

1. A method for preparing a biochip, comprising:
coating a chip with a first solution of biotin to form a biotin-coated chip, wherein the biotin in the first solution is in a first concentration ranging from 0.1 to 1 μg/ml;
providing the biotin-coated chip with a second solution of avidin to form an avidin/biotin-coated chip, wherein the avidin in the second solution is in a second concentration ranging from 0.1 to 100 μg/ml; and
providing the avidin/biotin-coated chip with a third solution of a biotinylated probe to form the biochip, wherein the biotinylated probed in the third solution is in a third concentration ranging from 1 to 3 μg/ml.

2. The method according to claim 1, wherein the chip is an extended gate field-effect transistor (EGFET).

3. The method according to claim 1, wherein the biotin in the first solution is in the first concentration of 0.1 μg/ml.

4. The method according to claim 3, wherein the avidin in the second solution is in the second concentration of 30 μg/ml.

5. The method according to claim 4, wherein the biotinylated probed in the third solution is in third the concentration of 1 μg/ml.

6. The method according to claim 1, wherein the biotin in the first solution is in the first concentration of 1 μg/ml.

7. The method according to claim 6, wherein the avidin in the second solution is in the second concentration of 100 μg/ml.

8. The method according to claim 7, wherein the biotinylated probed in the third solution is in the third concentration of 1 μg/ml.

9. The method according to claim 1, a ratio of the first concentration, the second concentration and the third concentration is 1:300:10.

10. The method according to claim 1, wherein the biotinylated probe is selected from the group consisting of a biotinylated PRRSV antibody, a biotinylated anti-Tau antibody and a biotinylated SARS-CoV-2 probe.

11. A biochip, prepared by a method comprising:
coating a chip with a first solution of biotin to form a biotin-coated chip, wherein the biotin in the first solution is in a first concentration ranging from 0.1 to 1 μg/ml;
providing the biotin-coated chip with a second solution of avidin to form an avidin/biotin-coated chip, wherein the avidin in the second solution is in a second concentration ranging from 0.1 to 100 μg/ml; and
providing the avidin/biotin-coated chip with a third solution of a biotinylated probe to form the biochip, wherein the biotinylated probed in the third solution is in a third concentration ranging from 1 to 3 μg/ml.

12. The biochip according to claim 11, wherein the chip is an extended gate field-effect transistor (EGFET).

13. The biochip according to claim 11, wherein the biotin in the first solution is in the first concentration of 0.1 μg/ml.

14. The biochip according to claim 13, wherein the avidin in the second solution is in the second concentration of 30 μg/ml.

15. The biochip according to claim 14, wherein the biotinylated probed in the third solution is in the third concentration of 1 μg/ml.

16. The biochip according to claim 11, wherein the biotin in the first solution is in the first concentration of 1 μg/ml.

17. The biochip according to claim 16, wherein the avidin in the second solution is in the second concentration of 100 μg/ml.

18. The biochip according to claim 17, wherein the biotinylated probed in the third solution is in the third concentration of 1 μg/ml.

19. The biochip according to claim 11, wherein a ratio of the first concentration, the second concentration and the third concentration is 1:300:10.

20. The biochip according to claim 11, wherein the biotinylated probe is selected from the group consisting of a biotinylated PRRSV antibody, a biotinylated anti-Tau antibody and a biotinylated SARS-CoV-2 probe.
